(19) United States
(12) Patent Application Publication
(10) Pub. No.: US 2011/0289802 A1
(43) Pub. Date: Dec. 1, 2011

(54) SHOE APPLIANCE WITH AN ORTHOPEDIC DEVICE

(76) Inventor: James G. Clough, Great Falls, MT (US)

(21) Appl. No.: 13/115,775

(22) Filed: May 25, 2011

Related U.S. Application Data
(60) Provisional application No. 61/348,518, filed on May 26, 2010.

(51) Int. Cl. A61F 5/14

(52) U.S. Cl. 36/140

(57) ABSTRACT

An orthopedic shoe appliance includes an insole, midsole or outersole of footwear; and one or more orthopedic devices disposed on the insole, midsole or outersole at one or more locations selected from the group consisting of a location under a big toe, a location under a medial longitudinal arch, a location under a lateral longitudinal arch, a location under a transverse metatarsal arch, and a location under and/or around a heel. Small wings may be attached to a part of the insole that will wrap around the medial and lateral aspects of the foot. A slit and hinge as well as changes in the flexibility of the materials allow the big toe joint to move independently of the other toes. Other pads may be applied to the top of the insole to allow for other corrections.

### CROSS REFERENCE TO RELATED APPLICATION

This claims the priority of U.S. Provisional Application Ser. No. 61/348,518, file on May 26, 2010, the disclosure of which is incorporated by reference in its entirety.

### BACKGROUND OF INVENTION

1. Field of the Invention

The present invention generally relates to shoe appliance (e.g., insoles, midsoles, and outersoles), particularly to shoe appliances including one or more orthopedic devices.

2. Description of Related Art

Shoes are worn for various purposes, such as protection of the foot from environmental hazards, support for the foot for normal foot function, comfort of the foot, etc. Supporting the foot to provide for normal foot function is a focus of many shoe manufacturers in their attempt to make shoes more comfortable. Different biomechanical theories have been proposed and adopted to improve the functional aspects of shoes.

FIG. 1 shows a typical view of the foot at rest on a typical shoe insole 7. The hallux 10 is resting on an upper planar surface 8 of the insole 7, which is parallel to a lower planar surface 1 of the insole 7. Without supporting the hallux 10, there is an increased likelihood that there will be excessive mobility of the medial arch area of the foot.

When a person ambulates or walks, a host of triplane motions occur. These motions are broadly termed pronation and supination. Pronation involves rotation of a joint or part in a forward direction or toward the midline of the body, while supination involves rotation of a joint or part in an outward direction or away from the midline of the body. When a person over-pronates or places too much force on the inside of a foot, excessive mobility of the medial arch area of the foot can result. A foot that stays pronated throughout the walking stance is inefficient at propelling the body forward. The instability resulting from over-pronation can lead to arch, foot, ankle, and/or leg pain, as well as postural problems due to excessive internal rotation of the leg. To propel the body forward in an efficient manner, the foot would supinate in midstance and become rigid with the joints assuming a close packed position.

Many different types of orthopedic corrective devices are available to address this problem. For example, U.S. Pat. No. 5,881,478, issued Mar. 16, 1999 to McMahon et al., teaches a shoe having a resilient sole, an upper secured to the sole, and a rockable member within a cavity in the sole. The rockable member being configured for side-to-side rocking in the sole cavity between a neutral position and a tilted position as the wearer's foot is moved relative to the sole between a neutral position and a tilted position.

U.S. Pat. No. 5,694,705, issued Dec. 9, 1997 to Alonso Coves, teaches an insole formed by the combination of two laminar bodies, one of split leather and the other of rubber material being provided with knobs forming support projections for the foot.

U.S. Pat. No. 4,408,402, issued Oct. 11, 1983 to Looney, teaches a supportive shoe or insert which provides increased support to specific areas of the foot during the first, second and third trimesters of pregnancy to compensate for changes in body weight and center of gravity. A pad, which can be a shoe insole, is provided with these specific areas of support.

U.S. Pat. No. 4,333,472, issued Jun. 8, 1982 to Tager, teaches compensatory-corrective orthopedic foot devices comprising of the construction and specific application of a series of differentially-sized geometrically-shaped and specifically configured, generally wedge-shaped, prosthetic devices that are utilized in the compensatory treatment of specific clinical structural biomechanical abnormalities of the human foot.

In U.S. Pat. Nos. 6,170,176, 6,874,258, and 6,938,363, inventor of the present invention disclosed shoe appliances, sold under the trade name of Cluffy Wedge®, that are effective in treating foot problems related to functional hallux limitus, first-ray insufficiency, and an unstable windlass mechanism.

These devices have proven useful in alleviating various foot problems. However, there still exists a need for other orthopedic devices that may provide other benefits.

### SUMMARY OF THE INVENTION

An aspect of the invention relates to orthopedic shoe appliances. An orthopedic shoe appliance in accordance with one embodiment of the invention includes an insole, midsole or outersole; and one or more orthopedic devices disposed on the insole, midsole, or outersole at one or more locations selected from the group consisting of a location under a big toe, a location under a medial longitudinal arch, a location under a lateral longitudinal arch, a location under a transverse metatarsal arch, and a location under and/or around a heel.

Another aspect of the invention relates to orthopedic shoe appliances having an insole, midsole, or outersole wherein the insole, midsole or outersole having a slit around a region under a big toe or just lateral to the big toe, such that the big toe is allowed to flex more or less independent from other toes. Such orthopedic shoe appliances may further include one or more orthopedic devices disposed on or in the insole, midsole or outersole at one or more locations selected from the group consisting of a location under a big toe, a location under or just proximal to the second and third metatarsals, a location under a medial longitudinal arch, a location under a lateral longitudinal arch, a location under a transverse metatarsal arch, and a location under and/or around a heel.

Other aspects of the invention will become evident from the following drawings and description.

### BRIEF DESCRIPTION OF THE DRAWINGS

FIG. 1 is a prior art figure illustrating the bone structure of a foot on a typical piece of footwear.

FIG. 2 is a top view of an insole having various locations for incorporating corrective pads in accordance with one embodiment of the invention.

FIG. 3 shows an illustration of an insole having a cutout for fitting an orthopedic device under a big toe in accordance with one embodiment of the invention.

FIG. 4A shows a side view of an insole in accordance with one embodiment of the invention. FIG. 4B shows the top view of the same insole.

FIG. 5 shows another insole having a slit separating the region under the big toe and the region under other toes in accordance with one embodiment of the invention.

FIG. 6 shows an insole having a patch that supports multiple locations under the foot in accordance with another embodiment of the invention. This patch is designed to allow the fourth and fifth metatarsals to bear more weight when the lateral arch of the foot becomes unstable.

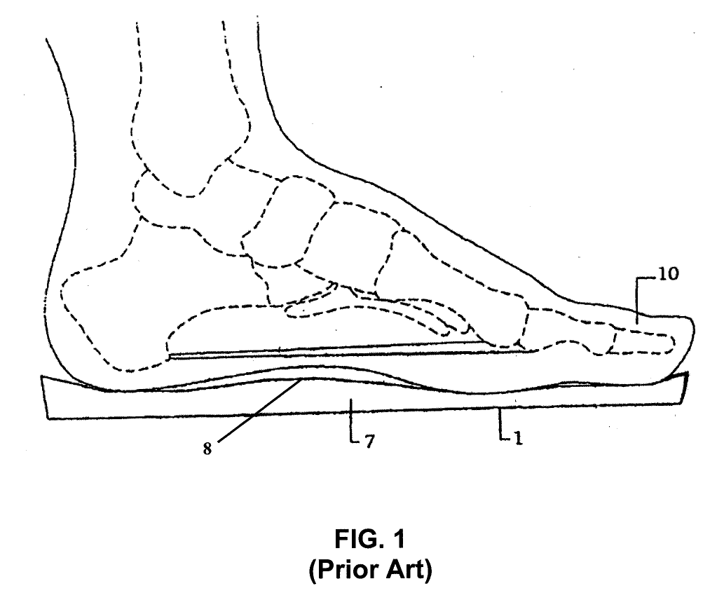

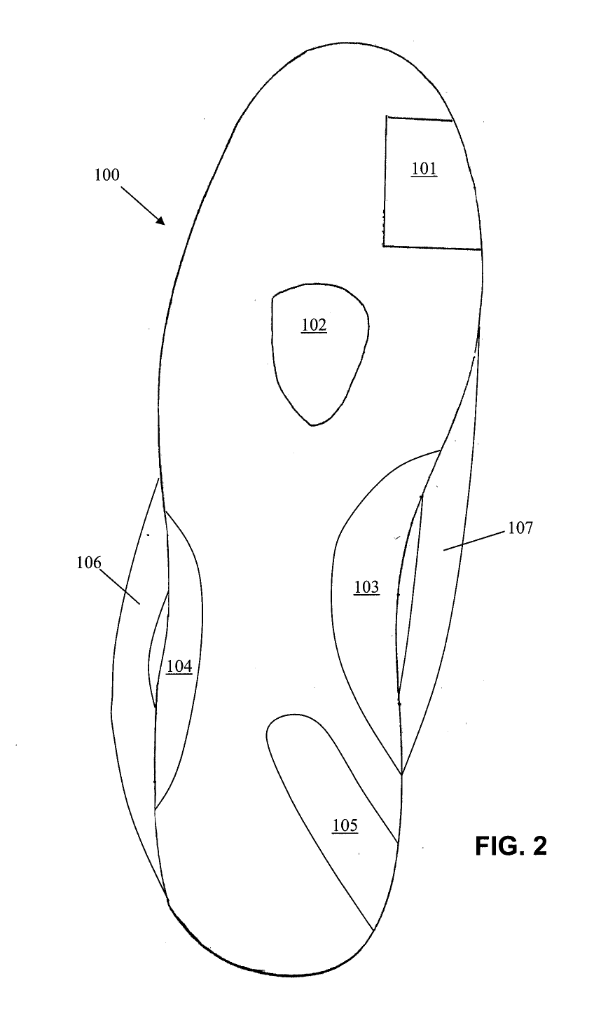

### DETAILED DESCRIPTION OF INVENTION

Embodiments of the invention relate to methods and apparatus for improving stability of a foot during ambulation. Some embodiments of the invention relate to insoles, midsoles, or outersoles having one or more orthopedic devices as accessories to alter properties of the insoles, midsoles and outersoles in specific areas. The following description may use “insole” to illustrate embodiments of the invention. However, this is for clarity of description. One skilled in the art would appreciate that the illustrated embodiments may also apply to midsoles and outersoles. The insoles may be used inside a shoe, as a footbed of a sandal, or in any suitable manner with a footwear. Alternatively, they may be used in the design of the footwear. The various orthopedic devices are placed at judiciously selected locations to provide different heights and/or different physical properties (e.g., flexibility or density) relative to the remaining parts of the footwear. Materials having different flexibility and/or density can allow different parts of a foot to flex to a different degree with respect to the neighboring regions of the foot. This relative flexibility may help to alleviate stress or pain during ambulation.

In accordance with embodiments of the invention, insoles, midsoles, or outersoles may include one or more orthopedic devices at the following locations for different purposes: under a big toe, under the medial longitudinal arch, under the lateral arch, under the transverse metatarsal arch, and/or to one side under the heel. By having different heights and/or different physical properties (flexibility or density), these orthopedic devices can prevent or remedy various problems associated with imperfect foot movements during ambulation.

In addition, when more than one of the orthopedic devices are used, synergistic effects may be realized. The gait cycle during ambulation may be broken into 3 phases. An insole of the invention may be designed to control the foot through all 3 phases of the gait cycle. When multiple pads are incorporated into an insole, midsole, or outersole design, together, the pads may work synergistically to accomplish the purpose of alleviate problems associated with imperfect foot during ambulation, to the benefit of the end user.

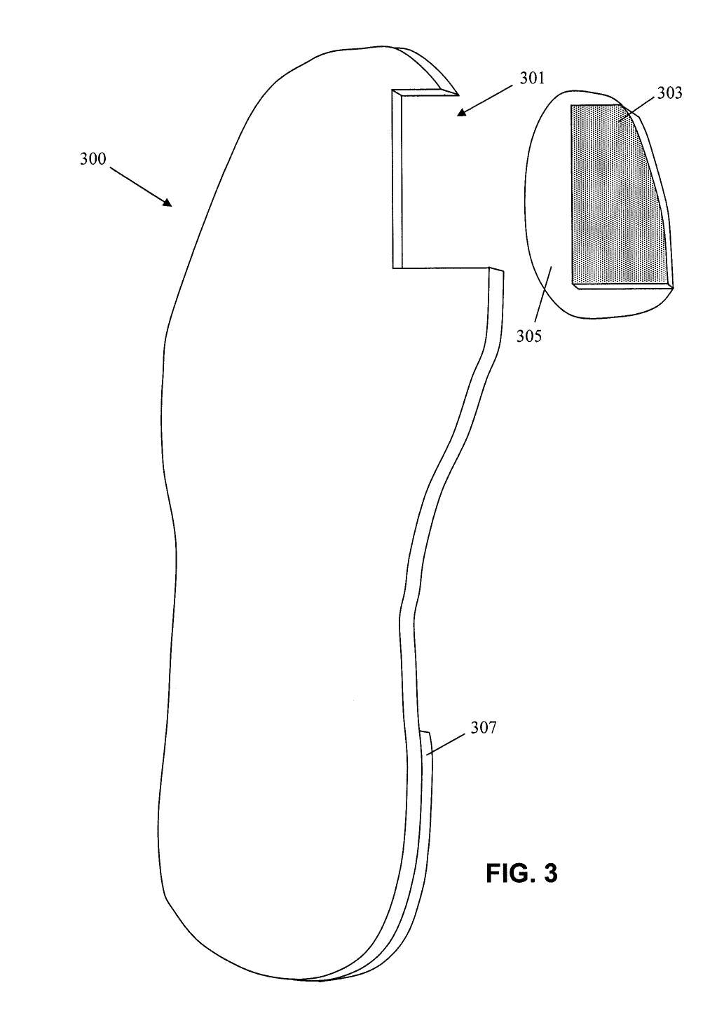

FIG. 2 shows a schematic of an insole 100 in accordance with one embodiment of the invention. The insole 100 may be made of any suitable material commonly employed for such purposes, such as flexible materials, leather, resilient foam-like materials, polyurethane gels, silicone gels, cork, thermoplastics, or combinations of various materials. The insole 100 may optionally include one or more small wings 106,107 that may fold up around the medial and lateral side of the foot when inserted into a shoe.

This insole 100 is shown with several locations where orthopedic devices may be incorporated: such as under a big toe 101, under a transverse metatarsal arch 102, under a medial longitudinal arch 103, under a lateral arch 104, and/or under a heel 105. As noted above, an insole of the invention may include one or more of orthopedic devices at one or more of these locations. The orthopedic devices may be made as an integral part of the insole, midsole, or outersole, or they may be made as “add-on” devices for attachment to these locations on an insole.

In alternative embodiments, locations 101-105 shown in FIG. 2 mark where cutouts may be made in an insole (see e.g., FIG. 3). In these alternative embodiments, one or more cutouts are made in an insole. Then, one or more orthopedic devices are made as separate pieces for fitting in these cutouts. Such embodiments have the advantages of convenience and flexibility in that different devices may be fit into the same insole to achieve different desired effects. For example, as a user's foot improves, less corrective effect may be needed. In such case, a user can simply put in the cutout a new device that would have a different property—e.g., heights, flexibility, or density.

The orthopedic devices may be fit in these cutouts with the aid of a fastening means. Any suitable fastening means may be used, such as glue, a hook and loop type of attachment system, or Velcro®. Alternatively, an orthopedic device may have a tab or a similar mechanism that would fit into or click into the cutouts on the insoles. A tab or a similar mechanism may simply be a configuration that is complementary to the configuration of the cutouts such that they can fit into the cutouts via compression fit.

As noted above, embodiments of the invention relate to insoles having one or more orthopedic devices at judiciously selected locations, such as under big toes (i.e., at location 101 in FIG. 2). In accordance with embodiments of the invention, a device may be configured as an add-on device for attachment on top or bottom of a regular insole, midsole or outersole or as a separate piece to fit into a cutout in an insole. The orthopedic devices to be used with insoles of the invention may be selected for various purposes—e.g., different heights, different flexibility, different elasticity, etc.

For example, in accordance with embodiments of the invention, some orthopedic devices may be selected for enhanced heights such as to lift the big toes during ambulation. Such orthopedic devices having different heights and/or inclinations may include, for examples, CluffyWedge® and devices disclosed in U.S. Pat. Nos. 6,170,176, 6,874,258, and 6,938,363, 7,849,610, or any other devices having similar properties. The description of these U.S. patents are incorporated by reference in their entirety. These devices would elevate the areas of the insoles, midsoles or outersoles under big toes to provide proper support for the big toes during ambulation. These devices may optionally be made as an integral part of the insoles. Alternatively, these devices may be made as accessory devices to be fit into cutouts in an insole.

When used as an add-on piece for fitting in a cutout, these devices may have different heights so that a proper height may be selected for a particular user, and a user may use different heights during different stages of correction.

In addition to height adjustment, some orthopedic devices of the invention may be selected for different flexibility, elasticity, and/or density to provide different degree or flex, yield, or compression when forces are exerted on them by foot during ambulation. One skilled in the art would know that any materials that can provide the desired physical properties may be used with embodiments of the invention. Such materials may include, for example, plastics, elastomers, foam, rubber (natural or synthetic), cork, gels, etc.

In addition to insoles having orthopedic devices under a big toe, some embodiments of the invention relate to insoles having orthopedic devices for placement under a medial longitudinal arch (i.e., location 103 in FIG. 2), which runs from front to back along the inside of the foot and is the most prominent foot arch. The medial longitudinal arch absorbs the majority of the shock of impact while walking, jumping or running. Therefore, an orthopedic device for placement under the medial longitudinal arch may be selected for its ability to cushion or buffer the impact.

Such devices for placement under the medial longitudinal arch may be made of a polymer material, such as foam, rubbers, elastomers, plastics, or the like. In addition, such a device may be made in various heights to accommodate different arch heights. Such devices may be made as integral parts of the insoles, midsoles, or outersoles. Alternatively, these devices may be made as add-on pieces for attachment on top or bottom of a regular insole or as separate pieces for fitting in a cutout in an insole.

Some embodiments of the invention relate to insoles, midsoles, or outersoles having orthopedic devices for placement under a lateral longitudinal arch (i.e., location 104 in FIG. 2), which runs parallel to the medial longitudinal arch, but is along the outer edge of the foot. Similar to the medial longitudinal arch, the lateral longitudinal arch also absorbs shock of impact while walking, jumping or running, and is important for those that tend to run their shoes to the outside of the midline of the body. Therefore, an orthopedic device for placement under the lateral longitudinal arch may be selected for its ability to cushion or buffer the impact. For example, these devices for placement under the lateral longitudinal arch may be made of a polymer material, such as foam, rubbers, elastomers, plastics, or the like. In addition, such devices may be made in various heights to accommodate different arch heights. Such devices may be made as integral parts of the insoles, midsoles or outersoles. Alternatively, these devices may be made as add-on pieces for attachment on top or bottom of a regular insole, midsole or outersole, or as separate pieces for fitting in a cutout in an insole.

Some embodiments of the invention relate to insoles, midsoles or outersoles having orthopedic devices for placement under a transverse metatarsal arch (i.e., location 102 in FIG. 2), which runs across around the neck of metatarsals from outside to inside. This arch also provides support and flexibility to the foot. Therefore, an orthopedic device for placement under the transverse metatarsal arch may be selected for its ability to cushion or buffer the impact. For example, these devices may be made of a polymer material, such as foam, rubbers, elastomers, plastics, or the like. In addition, such devices may be made in various heights to accommodate different arch heights. Such devices may be made as integral parts of the insoles, midsoles or outersoles. Alternatively, these devices may be made as add-on pieces for attachment on top or bottom of a regular insole, midsole or outersole or as separate pieces for fitting in a cutout in an insole, midsole or outersole.

Some embodiments of the invention relate to insoles, midsoles or outersoles having orthopedic devices for placement under and around the heel (i.e., location 105 in FIG. 2). The heel thus forms the posterior point of support that together with the balls of the large and little toes bear the brunt of the loads. Therefore, an orthopedic device for placement under the heel may be selected for its ability to cushion or correct improper foot stance.

For example, some embodiments of the invention relate to insoles, midsoles or outersoles having a heel plug of different densities to account for different foot types. A pronated foot may require a stiffer durometer material. In addition, some embodiments of the invention relate to insoles, midsoles or outersoles having heel lifts (shown as 307 in FIG. 3) for relieving heel pains or other foot pathology pains. Such heel plugs or heel lifts may be made as integral parts of the insoles, midsoles or outersoles. Alternatively, these devices may be made as add-on pieces for attachment on top of a regular insole, midsole or outersole or as separate pieces for fitting in a cutout in an insole, midsole, or outersole.

Embodiments of the invention also include insoles, midsoles or outersoles that incorporate two or more add-on pieces and/or cutouts discussed above, in any combination or permutation.

As noted above, an orthopedic device of the invention may be incorporated in an insole, midsole or outersole as an integral part. In preferred embodiments, these orthopedic devices may be separate pieces, either for attachment to the top or bottom surface of an insole, midsole or outersole or for fitting into cutouts in an insole, midsole or outersole. In other preferred embodiments, the orthopedic devices may be made for insoles, midsoles or outersoles with cutouts. Attachment of these separate orthopedic devices to an insole, midsoles or outersoles, may be via any means known in the art as noted above, such as glue or Velcro®, tabs, or compression fits with complementary devices and cutouts

FIG. 3 illustrates a method for attaching an orthopedic device to an insole with a cutout using a Velcro®. In this example, this insole 300 has a big toe cutout 301 and the under side of the insole, at least around the cutout 301, may be provided with Velcro® for attachment of an orthopedic device 303 (such as a Cluffy Wedge®) using Velcro®. The orthopedic device 303 is affixed with a piece of Velcro® 305, which may be used to secure the orthopedic device 303 to the insole 300 using the Velcro® on the underside (not shown) of the insole. The hook and loop parts of Velcro® may be located on the orthopedic device and the insoles, midsoles or outersoles, respectively, or vice versa. The orthopedic device 303 fits in the cutout 301 in the insole 300 in the area where the big toe would rest. The orthopedic device 303 provides a means to elevate the big toe from the top surface of the insole 300. The heights of the orthopedic device 303 may be varied to fit the need of a user. In addition, the orthopedic device 303 may optionally have a slanted top side such that its top surface will form an angle (e.g., 0-60 degrees) relative to the top surface of the insole 300.

The orthopedic device 303 will function to stabilize the first metatarsal against ground reactive forces and limit the displacement of the first metatarsal. Thus, the first metatarsal will plantarflex more easily through the late midstance and propulsive phases of a gait. Placing the plantar aponeurosis on stretch will result in a retrograde effect at stabilizing the joints that are more proximally referred to as the midtarsal and subtalar joints. They will enjoy improved joint congruity, and alignment of the foot in relationship to the leg during ambulation will also improve. When the first metatarsal phalangeal joint is able to dorsiflex 0-60 degrees, normal plantarflexion of the first metatarsal is possible and the normal mechanics of the gait cycle are not disrupted during ambulation. The orthopedic device 303 can provide such a 0-60 degree dorsiflexion of the first metatarsalphalangeal joint of the foot.

FIG. 3 shows an example of attaching an orthopedic device in a big-toe cutout of an insole. One skilled in the art would appreciate that similar methods may be used to attach orthopedic devices at other locations (e.g., locations 102-105 shown in FIG. 2). Embodiments of the invention may have any number, for example from 1 to 5, of orthopedic devices with any combination or permutation of devices described above. A combination use of these devices (i.e., two or more) may produce synergistic effects.

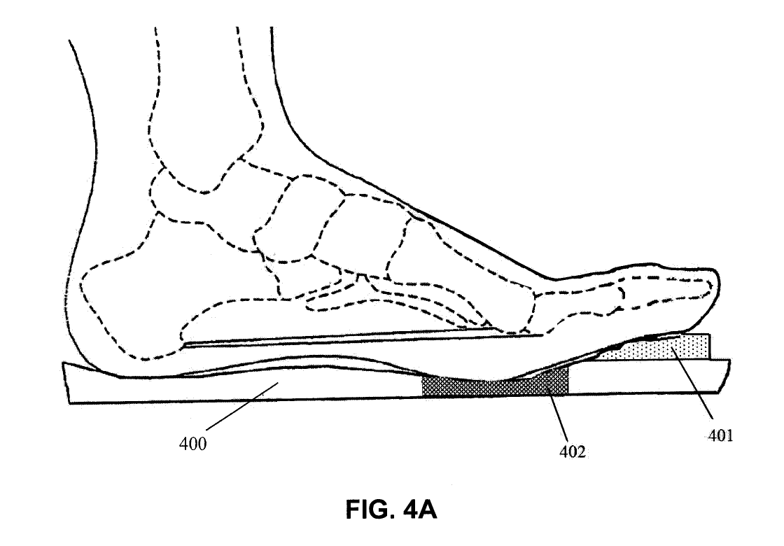

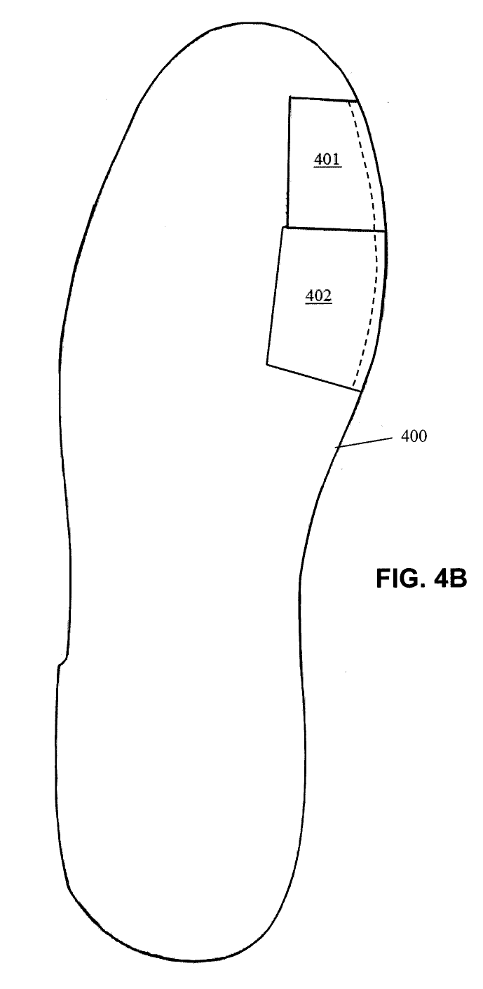

FIG. 4A shows a side view of some embodiments of the invention. FIG. 4B shows a top view of such an insole. As shown, an insole, midsole or outersole 400 of the invention may be equipped with a wedge 401 under the big toe and/or a patch 402 under the first metatarsal head, herein the patch 402 is made of a material different from the remaining part of the insole, midsole or outersole such that the patch 402 would allow the first metatarsal head regions to flex to a different extent as compared to other regions. The different material, for example, may have a lower durometer (hardness) to allow the first metatarsal head to flex downward more easily when the big toe flex upward in dorsiflexion. Dorsiflexion of a big toe accomplishes several significant biomechanical consequences, which have a net effect of providing a supinatory position of a subtalar and midtarsal joint, as well as pronation of a longitudinal midtarsal joint.

In some other embodiments, the patch 402 made be made of a material with a higher durometer to provide more support for the first metatarsal head region, if such correction is needed.

The wedge 401 allows the big toe to flex upward more during ambulation. The wedge 401 and the patch 402 may be used in concert to achieve a better effect, including a synergistic effect. Note that the outer edge of the wedge 401 and/or the patch 402 may be configured to extend all the way to the edge of the insole, midsole or outersole 400. Alternatively, the wedge 401 and/or the patch 402 may not extend all the way to the edge of the insole, midsole or outersole 400, as illustrated in dotted lines in FIG. 4B.

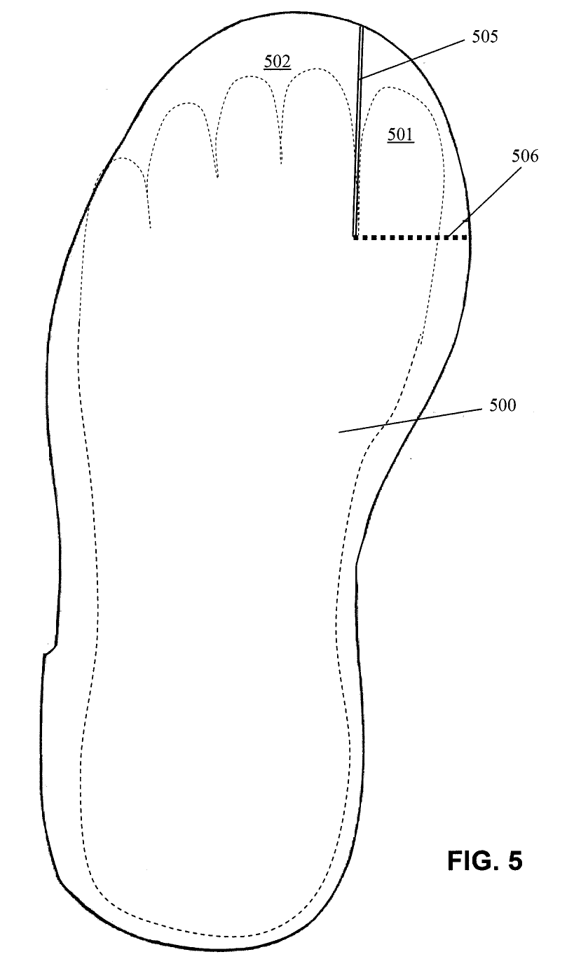

In accordance with some embodiments of the invention, a different degree of mobility of the big toe may be achieved with an insole, midsole and/or outersole having a slit to allow the region under the big toe to move independently of the remaining regions of the foot (e.g., second to fifth toes). FIG. 5 shows one such example. As shown in FIG. 5, an insole 500 contains a cut (slit) 505 that separates the region 501 under the big toe from the remaining part 502 of the sole under the second-to-fifth toes. In addition to using the slit 505 to allow independent movement of the region under the big toe, one can also incorporate different materials in the regions 501 and/or 502 to further differentiate the relative movements of these parts of the foot. For example, by using a material with a lower durometer in region 501 and a higher durometer material in region 502, the big toe would move upward more than the other toes during ambulation—i.e., the big toe would dorsiflex relative to other toes. The different materials may be used in concert with the slit 505 to achieve synergistic effects.

Note that the slit 505 embodiment shown in FIG. 5 may further incorporate a hinge 506 that would allow region 501 to flex more easily relative to other parts of the insole, midsole, or outersole. The hinge 506 may be a partial cut (score) along the dotted line as indicated. Alternatively, the hinge 506 may be made of a more flexible material. One skilled in the art would appreciate that any mechanism that allows for more flexibility may be used. Furthermore, in some embodiments, a patch under the first metatarsal head (shown as 402 in FIG. 4B) may be used with the allotted embodiment shown in FIG. 5. A combination of the slit 505 and a patch of different durometer may provide enhanced correction effects.

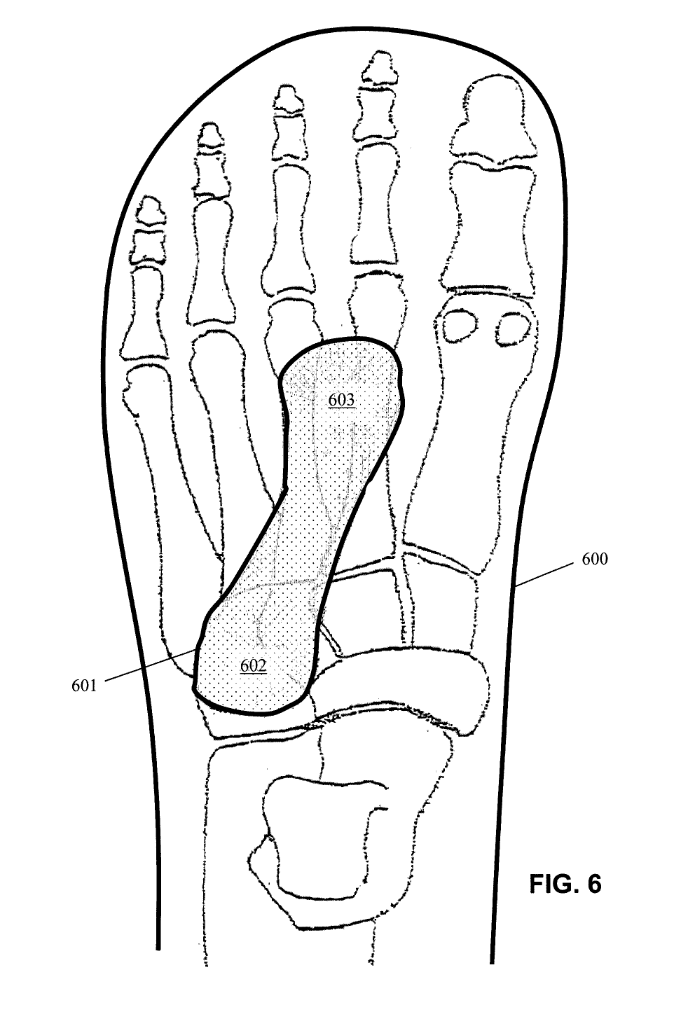

As shown in FIG. 6, an insole 600 may have a patch 601 disposed on the top surface thereof. Such embodiments can be used to correct lateral column instabilities. The patch 601 may provide different heights relative to the top surface of the insole 600 to achieve different support to the foot during ambulation. Furthermore, different areas of the patch 601 may be configured to have different heights. For example, the area 602 may have a different height and/or durometer to provide different support to the foot under the area around the cuboid, and area 603 may have a different height and/or durometer to provide different support under the foot in the region around the second and third metatarsals.

Embodiments of the invention may have one or more of the following advantages. Insoles of the invention may have one or more orthopedic devices for correcting various foot problems. These different orthopedic devices are attached to different areas in an insole. These devices may be selected for different dimensions or different materials to provide the needed correction to the foot during ambulation. Some devices of the invention may be separate pieces such that they can be attached and removed from the insole and a different device with a different property (e.g., different heights or different flexibility or density) may be substituted as needed.

While the invention has been described with respect to a limited number of embodiments, those skilled in the art, having the benefit of this disclosure, will appreciate that other embodiments that do not depart from the scope of the invention as disclosed herein can be devised. Accordingly, the scope of the invention should not be limited only by the attached claims.

1. An orthopedic shoe appliance, comprising:
an insole, midsole or outersole; and
one or more orthopedic devices disposed on or in the insole, midsole or outersole at one or more locations selected from the group consisting of a location under a big toe, a location under second and third metatarsals, a location under a medial longitudinal arch, a location under a lateral longitudinal arch, a location under a transverse metatarsal arch, and a location under and/or around a heel.

2. The orthopedic shoe appliance of claim 1, wherein the one or more orthopedic devices are configured to provide different heights and/or flexibility.

3. The orthopedic shoe appliance of claim 1, wherein one of the one or more orthopedic devices is configured to provide a different height.

4. The orthopedic shoe appliance of claim 3, wherein the one orthopedic device providing a different height is a the location under the big toe and has a slant top surface having an incline angle of 0-60 degrees relative to a top surface of the insole.

5. The orthopedic shoe appliance of claim 1, wherein the insole, midsole or outersole comprises one or more cutouts and the one or more orthopedic devices are configured to fit in the one or more cutouts.

6. The orthopedic shoe appliance of claim 5, wherein the one or more orthopedic devices are configured to provide different heights and/or flexibility.

7. The orthopedic shoe appliance of claim 5, wherein one of the one or more orthopedic devices is configured to provide a different height.

8. The orthopedic shoe appliance of claim 7, wherein the one orthopedic device providing a different height is at the location under the big toe and has a slant top surface having an incline angle of 0-60 degrees relative to a top surface of the insole.

9. An orthopedic shoe appliance, comprising an insole, midsole or outersole wherein the insole, midsole or outersole having a slit around a region under a big toe such that the big toe is allowed to flex independently of other toes.

10. The orthopedic shoe appliance of claim 9, further comprising a hinge to allow the region under the big toe to facilitate better motion of the big toe.

11. The orthopedic shoe appliance of claim 9, further comprising one or more orthopedic devices disposed on or in the insole, midsole or outersole at one or more locations selected from the group consisting of a location under a big toe, a location under second and third metatarsals, a location under a medial longitudinal arch, a location under a lateral longitudinal arch, a location under a transverse metatarsal arch, and a location under and/or around a heel.

12. The orthopedic shoe appliance of claim 9, wherein the one or more orthopedic devices are configured to provide different heights and/or flexibility.

13. The orthopedic shoe appliance of claim 9, wherein one of the one or more orthopedic devices is configured to provide a different height.

14. The orthopedic shoe appliance of claim 13, wherein the one orthopedic device providing a different height is at the location under the big toe and has a slant top surface having an incline angle of 0-60 degrees relative to a top surface of the insole, midsole or outersole.

15. The orthopedic shoe appliance of claim 9, wherein the insole, midsole or outersole comprises one or more cutouts and the one or more orthopedic devices are configured to fit in the one or more cutouts.

16. The orthopedic shoe appliance of claim 15, wherein the one or more orthopedic devices are configured to provide different heights and/or flexibility.

17. The orthopedic shoe appliance of claim 15, wherein one of the one or more orthopedic devices is configured to provide a different height.

18. The orthopedic shoe appliance of claim 17, wherein the one orthopedic device providing a different height is at the location under the big toe and has a slant top surface having an incline angle of 0-60 degrees relative to a top surface of the insole, midsole or outersole.
